(19) United States
(12) Patent Application Publication
(10) Pub. No.: US 2026/0194537 A1
(43) Pub. Date: Jul. 9, 2026

(54) NON-INVASIVE METHODS OF DIAGNOSING OR MONITORING STEATOHEPATITIS

(71) Applicants: INSERM (INSTITUT NATIONAL DE LA SANTÉ ET DE LA RECHERCHE MÉDICALE), Paris (FR); UNIVERSITÉ DE RENNES, Rennes (FR); ECOLE DES HAUTES ETUDES EN SANTÉ PUBLIQUE (EHESP), Rennes (FR); CHU DE RENNES, Rennes (FR)

(72) Inventors: Michel SAMSON, Rennes (FR); Romain MOIRAND, Rennes Cedex 9 (FR); Arnaud CHALIN, Guipry (FR)

(21) Appl. No.: 19/130,848

(22) PCT Filed: Nov. 16, 2023
(86) PCT No.: PCT/EP2023/082033

§ 371 Date: May 16, 2025

(30) Foreign Application Priority Data
Nov. 17, 2022 (EP) 22306690.3

(51) Int. Cl. G01N 33/68

(52) U.S. Cl. CPC G01N 33/6893; G01N 2800/085; G01N 2800/52

(57) ABSTRACT

The invention relates to the field of diagnosing steatohepatitis, in particular alcoholic steatohepatitis. By following studies on a cohort of nearly 200 patients, the inventors showed that four or five specific, readily available markers, when combined in a function, make it possible, by themselves, to highly accurately diagnose steatohepatitis at the early stages of the disease, when the subject is still asymptomatic. In particular, the invention relates to a non-invasive, highly performant method of diagnosing steatohepatitis in a subject, which combines in a function the values of the age, the blood concentration of cytokeratin 18 and at least two of: the body mass index, the blood concentration of α-2-macroglobulin and a data representative of liver stiffness as obtained by an ex vivo imaging technique. The method of the invention makes it possible to identify asymptomatic patients that may then benefit from potential therapeutics and/or be motivated to reduce alcohol consumption.

### FIELD OF THE INVENTION

The invention relates to an in vitro method of diagnosing alcoholic steatohepatitis in a subject, as well as to an in vitro method of monitoring the evolution of such a disease in a subject. These methods are particularly useful for non-invasive diagnostic of asymptomatic alcoholic steatohepatitis.

### BACKGROUND OF THE INVENTION

Steatohepatitis is a fatty liver disease associated with liver-damaging inflammation. It can be either alcohol-related (it is then referred to as ASH, for Alcoholic SteatoHepatitis), or non-alcohol-related (it is then referred to as NASH, for Non-Alcoholic SteatoHepatitis), ASH and NASH being different diseases.

An estimated 607,000 deaths due to liver cirrhosis and 22.2 million disability-adjusted life years worldwide in 2016 have been attributed to alcohol-related liver disease (ALD) (Organization WH. Global Status Report on Alcohol and Health 2018. 2019). ALD comprises a spectrum of diseases that are classified histologically and follow a well-recognized pattern of disease progression (Seitz et al., 2018). Most patients present with alcoholic fatty liver (AFL) and will have a benign clinical course. Some individuals will progress to alcoholic steatohepatitis (ASH), with inflammation and hepatocyte injury. ASH is mostly asymptomatic and may progress slowly, leading to progressive fibrosis and cirrhosis (European Association for the Study of the Liver, 2018). A few patients will develop alcoholic hepatitis (AH), characterized by the rapid onset of jaundice (Lucey, 2009). ALD is mild and asymptomatic for years but, if cirrhosis occurs, patients can worsen rapidly or develop hepatocellular carcinoma (European Association for the Study of the Liver, 2018).

As regards diagnostic, the presence of advanced fibrosis is an important prognostic factor (de Franchis et al., 2022). Being able to distinguish between patients with ASH and those with AFL could also have beneficial therapeutic implications. Indeed, the presence of ASH has been associated with a greater progression of liver disease towards advanced fibrosis and complications (Chedid et al., 1991). In a cohort of patients with subsequent biopsies, ASH was associated with the highest risk of fibrosis progression compared to steatosis and the extent of fibrosis (Mathurin et al., 2007). The first line of treatment in ALD is alcohol cessation, since persistent alcohol intake is an independent predictive factor of death in ALD (Lackner et al., 2017). ASH identification could be used as a supplementary motivation for the patient to reduce alcohol consumption, as ALD screening in hazardous drinkers has been shown to have a potential positive impact on alcohol consumption (Eyles et al., 2013). More generally, it could trigger the implementation of pharmacological interventions to stop the disease progression. Indeed, steatohepatitis can be reversed, or at least its consequences can be limited, provided that it is managed early. This implies being able to diagnose the disease at an early stage after its appearance.

The diagnostic of alcoholic steatohepatitis, in particular asymptomatic ASH, is currently based on liver biopsy, an invasive, costly procedure with non-negligible morbidity (Piccinino, 1986).

In order to avoid performing liver biopsy, several non-invasive methods for diagnosing liver diseases have been proposed by the prior art. These methods combine, in a mathematical function, the values of several biological markers and clinical markers measured on the patient, in order to obtain an end value reflecting the probability of the patient having the disease.

The so-called AshTest® (Rudler et al., 2015) and NashTest® (Harrison et al., 2020) are currently proposed for, respectively, ASH and NASH diagnostic. These non-invasive diagnostic methods both require a high number of markers to be measured on the patient (respectively, 9 and 11). Moreover, they are designed for diagnosing severe forms of steatohepatitis.

The publications Feldstein et al., Hepatology, 50 (4), 2009, 1072-1078, Liebig et al., Clinical and Translational Gastroenterology, 10 (8), 2019, e00066 and Yuping et al. Disease Markers, 2022, 2022, 1-12, all disclose non-invasive methods of diagnosing Non-Alcoholic SteatoHepatitis (NASH). WO 2018/073618 discloses a method of diagnosing alcoholic hepatitis (AH). The publication Yilmaz, Alimentary Pharmacology & Therapeutics, 30 (11-12), 2009, 1103-1109 discloses that fragments of CK-18 are useful biomarkers for diagnosing NASH. None of these documents provides a method for diagnosing alcoholic steatohepatitis (NASH) in a subject.

There still remains a need for a satisfactory non-invasive method for accurately diagnosing steatohepatitis, more particularly alcoholic steatohepatitis, at an early stage after its appearance, in particular in asymptomatic patients.

The invention aims to fulfill such a need, by proposing a non-invasive method for diagnosing steatohepatitis, more particularly alcoholic steatohepatitis, which is performant even at the early stages of the disease, when the subject is still asymptomatic.

Further objectives of the invention are that this method is easy and rapid to implement, and that it allows for a highly accurate diagnostic based on a smaller number of markers than the methods of the prior art.

An additional objective of the invention is that this method only uses markers that are readily available for the type of patients concerned, i.e., markers which are currently analyzed during the standard clinical evaluation of such patients, or are very easy to analyze in the course of such a clinical evaluation.

### SUMMARY OF THE INVENTION

The inventors have now discovered that these objectives can be achieved by using a small number of specific biomarkers associated with the disease and physical characteristics of the subject, all of which are easily accessible or available from the results of medical examination commonly carried out on the subject when steatohepatitis is suspected. More particularly, it has been discovered by the inventors that a highly accurate diagnostic of steatohepatitis can be obtained using a number as low as four or five markers, when combining specific blood biochemical marker(s), clinical marker(s) and, optionally, a data issued from an imaging technique.

Thus, according to a first aspect, the invention relates to an in vitro method of diagnosing steatohepatitis, more particularly alcoholic steatohepatitis (ASH) in a subject, comprising steps of:
a/ collecting for said subject:
   a value of the concentration of cytokeratin 18, the fragment M65 or the fragment M30 thereof, and optionally a value of the concentration of α-2-macroglobulin, in an isolated blood sample obtained from said subject,
   optionally, a data representative of liver stiffness as obtained by an ex vivo imaging technique,
   the value of the age, and optionally the value of the body mass index, of the subject,
b/ combining in a mathematical function the value of the age of the subject, the concentration in said blood sample of cytokeratin 18, the fragment M65 or the fragment M30 thereof, and at least two of: the concentration of α-2-macroglobulin in said blood sample, the data representative of liver stiffness and the value of the body mass index of the subject,
in order to obtain a score,
   c/ comparing said score to a predetermined first reference value,
   d/ and concluding that the subject has steatohepatitis, and more particularly alcoholic steatohepatitis, when said score is higher than or equal to said first reference value.

Another aspect of the invention concerns an in vitro method of monitoring steatohepatitis, more particularly alcoholic steatohepatitis, in a subject, comprising:
a/ at a first specific time of the disease then at a second specific time of the disease, collecting for said subject: a value of the concentration of cytokeratin 18, the fragment M65 or the fragment M30 thereof, and optionally a value of the concentration of α-2-macroglobulin, in an isolated blood sample obtained from said subject; optionally, a data representative of liver stiffness as obtained by an ex vivo imaging technique; and the value of the age, and optionally the value of the body mass index, of the subject,
b/ for each of the first specific time of the disease and the second specific time of the disease, combining in a mathematical function the value of the age of the subject, the concentration in said blood sample of cytokeratin 18, the fragment M65 or the fragment M30 thereof, and at least two of: the concentration of α-2-macroglobulin in said blood sample, the data representative of liver stiffness and the value of the body mass index of the subject, in order to obtain a score,
c/ comparing the score obtained for the second specific time of the disease with the score obtained for the first specific time of the disease,
d/ and concluding that the disease got worse when the score obtained for the second specific time of the disease is higher than the score obtained for the first specific time of the disease.

A further aspect of the invention is an in vitro method of monitoring the treatment of steatohepatitis, more particularly alcoholic steatohepatitis, in a subject comprising:
a/ at a time before the start of the treatment, then at a specific time during or after the treatment, collecting for said subject a value of the concentration of cytokeratin 18, the fragment M65 or the fragment M30 thereof, and optionally a value of the concentration of α-2-macroglobulin, in an isolated blood sample obtained from said subject; optionally, a data representative of liver stiffness as obtained by an ex vivo imaging technique; and the value of the age, and optionally the value of the body mass index, of the subject,
b/ for each of the time before the start of the treatment and the specific time during or after the treatment, combining in a mathematical function the value of the age of the subject, the concentration in said blood sample of cytokeratin 18, the fragment M65 or the fragment M30 thereof, and at least two of: the concentration of α-2-macroglobulin in said blood sample, the data representative of liver stiffness and the value of the body mass index of the subject, in order to obtain a score,
c/ comparing the score obtained for the specific time during or after the treatment with the score obtained for the time before the start of the treatment,
d/ and concluding that the treatment is efficient when the score obtained for the specific time during or after the treatment is lower than the score obtained for the time before the start of the treatment.

Another object of the invention relates to a method of treating steatohepatitis, more particularly alcoholic steatohepatitis, with an appropriate treatment, in a subject in which steatohepatitis, more particularly alcoholic steatohepatitis, has been diagnosed by an in vitro method of diagnosing steatohepatitis, more particularly alcoholic steatohepatitis, according to the invention.

A further object of the invention is a method of treating steatohepatitis, more particularly alcoholic steatohepatitis, in a subject, comprising:
a/ collecting for said subject: a value of the concentration of cytokeratin 18, the fragment M65 or the fragment M30 thereof, and optionally a value of the concentration of α-2-macroglobulin, in an isolated blood sample obtained from said subject; optionally, a data representative of liver stiffness as obtained by an ex vivo imaging technique; the value of the age, and optionally the value of the body mass index, of the subject,
b/ combining in a mathematical function the value of the age of the subject, the concentration in said blood sample of cytokeratin 18, the fragment M65 or the fragment M30 thereof, and at least two of: the concentration of α-2-macroglobulin in said blood sample, the data representative of liver stiffness and the value of the body mass index of the subject, in order to obtain a score,
c/ comparing said score to a predetermined first reference value,
d/ and if said score is higher than or equal to said first reference value, treating the subject with an appropriate treatment.

### DETAILED DESCRIPTION OF THE INVENTION

As used herein, the term steatohepatitis refers to a fatty liver disease characterized by the presence of hepatic steatosis and inflammation with hepatocyte injury, with or without fibrosis.

A target of the invention, to which it is particularly well-adapted, is alcoholic steatohepatitis, in particular asymptomatic alcoholic steatohepatitis.

The invention can however also be applied to non-alcoholic steatohepatitis, for example associated with metabolic dysfunction.

### Diagnostic

A first aspect of the invention relates to an in vitro, non-invasive, method of diagnosing steatohepatitis, more particularly alcoholic steatohepatitis, in a subject.

The term “diagnosing” herein means identifying the presence of the disease and/or assessing the severity and/or the stage thereof.

The diagnostic method of the invention comprises:
a/ a first step of collecting for the subject:
   value(s) representative of the circulating amount of specific biochemical marker(s) in the blood, serum or plasma of the subject, more particularly a value of the concentration of cytokeratin 18, the fragment M65 or the fragment M30 thereof, and optionally a value of the concentration of α-2-macroglobulin, as measured in an isolated blood sample obtained from the subject,
   optionally, a data representative of liver stiffness as obtained by an ex vivo imaging technique, for example by performing ex vivo transient elastography on the liver of the subject, and
   the value of the age of the subject, and optionally the value of the body mass index of the subject,
b/ combining in a mathematical function, as the variables of the function, the values thus collected of the age of the subject, the concentration of cytokeratin 18 or the fragment M65 or the fragment M30 thereof in said blood sample, and at least two of the three following variables: the concentration of α-2-macroglobulin in said blood sample, the data representative of liver stiffness and the value of the body mass index of the subject,
in order to obtain a diagnostic score, which is indicative of the presence or absence of steatohepatitis, more particularly alcoholic steatohepatitis, in the subject, and, more precisely, reflects the probability for the subject to have steatohepatitis, more particularly alcoholic steatohepatitis.

The method of the invention then comprises steps of:
c/ comparing the score thus obtained with a predetermined first reference value,
d/ and, when this score is higher than or equal to this first reference value, deducing that the subject has steatohepatitis, more particularly alcoholic steatohepatitis.

As used herein, the phrase “blood sample” means a whole blood sample obtained from the subject, or a sample of serum or a sample of plasma that have been recovered from a whole blood sample.

All the markers included in the function of the invention are well-known by the person skilled in the art, and have been described in the prior art as associated with liver disease.

Cytokeratin 18 (K18) represents the major intermediate filament protein in liver cells (Romano et al., 1986). K18 is the main component of Mallory-Denk bodies (MDBs), which are a hallmark of alcoholic steatohepatitis (Stumptner et al., 2000). During cell death, K18 is cleaved by caspases and exposes two epitopes: M30, a marker of apoptosis, and M65, a marker of overall cell death (Leers et al., 1999). Alpha-2-macroglobulin (α2M) is a large proteinase inhibitor mainly produced by the liver, and elevated circulating levels have been shown to be associated with advanced fibrosis and cirrhosis in ALD (Chrostek et al., 2014). The measurement of liver stiffness by an ex vivo imaging technique, in particular by ex vivo transient elastography, in hepatic tissues has been widely validated for the diagnostic of advanced fibrosis from multiple chronical etiologies, including alcohol-related (Moreno et al., 2019). It is known that liver inflammation is an independent factor associated with higher risk of fibrosis progression (Mathurin et al., 2007). The body mass index (BMI) is known to markedly increase the presence of steatosis in ALD (Bellentani et al., 2001).

However, nothing in the prior art suggested that such a specific combination of markers as that of the invention could make it possible to predict with a particularly high accuracy the probability of a subject having steatohepatitis, and more particularly alcoholic steatohepatitis. This high accuracy has in particular been verified in a biopsy-controlled study conducted in a large homogeneous population of asymptomatic heavy drinkers initiating an alcohol detoxification protocol. In such a study, the score based on the best function according to the invention, combining the four or five variables described above, was able to accurately classify more than two-thirds of patients diagnosed as having alcoholic steatohepatitis by liver biopsy, with excellent diagnostic performances. The method of the invention advantageously brings new diagnostic opportunities and openings new possibilities of significantly impacting the management of patients having asymptomatic alcoholic steatohepatitis.

As used herein, the term “accuracy” means the proportion of patients which are correctly classified by a diagnostic method.

The performance of a diagnostic test is generally assessed by drawing a Receiving Operating Characteristic (ROC) curve and measuring the Area Under the Curve (AUC). The ROC curve is drawn by plotting the sensitivity versus (1-specificity) after classification of the patients on a scale of 0 to 1, according to the accuracy of the results obtained by the diagnostic method. The closer the AUC value is to 1, the higher the sensitivity and specificity of the method and the more performant the diagnostic method.

“Sensitivity” herein means, in a conventional manner, the probability of the diagnostic method to identify as positive the subjects having the disease, i.e., to identify the true positives. “Sensibility” means the probability of the diagnostic method not to identify as positive the subjects not having the disease, i.e., not to identify the true negatives.

Moreover, as used herein, the “Positive predictive value” (PPV) is the probability for a subject of having the disease when the result of the diagnostic method is positive. The “Negative predictive value” (NPV) is the probability for a subject of not having the disease when the result of the diagnostic method is negative.

In the above-mentioned study, the method of the invention made it possible to obtain a diagnostic of alcoholic steatohepatitis with an AUC as high as 0.93, with a 95% Confidence Interval (CIs) of [0.89-0.97], which demonstrates a particularly high performance.

The reference value used in the method of the invention is preferably a cut-off value. It is well within the skills of the person skilled in the art to establish such a cut-off value for the statistical diagnostic method of the invention, depending on the required level of selectivity and specificity for the diagnostic and benefit/risk balance associated with the disease. Such a determination may be carried out either experimentally or theoretically.

In particular embodiments of the invention, the method also comprises comparing the score obtained by the mathematical function of the invention with a predetermined second reference value, and concluding that the subject does not have steatohepatitis, more particularly alcoholic steatohepatitis, when this score is lower than or equal to this second reference value.

In particular embodiments of the invention, the predetermined first reference value and the predetermined second reference value are such that:
the positive predictive value is higher than 90%, preferably higher than 95%, above the first reference value,
the negative predictive value is higher than 90%, preferably higher than 95%, below the second reference value.

These reference values allow a classification of the subject into one of the three following groups: with steatohepatitis (when the calculated score is higher than or equal to the first reference value, so-called upper cut-off value)/without steatohepatitis (when the calculated score is lower than or equal to the second reference value, so-called lower cut-off value)/uncertain (when the calculated score is between the first reference value and the second reference value, these values not included).

The diagnostic method of the invention, the result of which is based on statistics, owing to its very good reliability, provides a very helpful information for the physician for making his clinical diagnostic and, where appropriate, prescribing steps to be taken for the management of the disease. It can help early identification of asymptomatic patients with high risk of liver disease progression, and it can for example help practitioners to motivate those patients to, depending on the type of steatohepatitis, stop or reduce alcohol consumption and/or adopt a more appropriate diet and/or initiate suitable drug treatment. More generally, it advantageously allows a better monitoring and global disease management.

The subject on which the diagnostic method of the invention is applied is preferably a mammal, such as a feline, a canine, an equine or a primate. In preferred embodiments of the invention, the subject is a human.

The subject may as well be afflicted by steatohepatitis as he may be healthy. By “healthy”, it is herein intended to mean that the subject is not afflicted by steatohepatitis, more particularly alcoholic steatohepatitis, whether or not he is afflicted by another disease.

In particular embodiments of the invention, the subject is asymptomatic with respect to steatohepatitis, more particularly alcoholic steatohepatitis.

In particular embodiments of the invention, the steatohepatitis targeted by the method of the invention is alcoholic steatohepatitis (ASH). It can otherwise be non-alcoholic steatohepatitis (NASH).

Preferably, both the concentration of α-2-macroglobulin in the blood sample obtained from the subject and the data representative of liver stiffness, as obtained by an ex vivo imaging technique, in particular by transient elastography, are included in the mathematical function of the invention.

In particularly preferred embodiments of the invention, the concentration of α-2-macroglobulin in said blood sample, the data representative of liver stiffness, as obtained by an ex vivo imaging technique, in particular by ex vivo transient elastography, and the value of the body mass index of the subject, are all three included in the mathematical function of the invention. In particular, it has been observed by the inventors that including in the function the result of the ex vivo imaging technique measurement, the value of the body mass index of the subject and a value representative of the concentration of α-2-macroglobulin circulating in the blood of the subject, significantly improves the accuracy of the probabilistic scoring system of the invention for the diagnostic of alcoholic steatohepatitis.

The mathematical function of the invention can include other variables than those described above. These variables can in particular be relative to other biological markers and/or physical characteristics of the subject. In such particular embodiments of the invention however, preferably, the mathematical function does not include any data associated with the marker bilirubin, such as bilirubin concentration.

In preferred embodiments of the invention, no other variable than those described above are included in the mathematical function, which then comprises no more than four or five variables, depending on the inclusion or not of the α-2-macroglobulin and/or the data representative of liver stiffness and/or the value of the body mass index of the subject.

The diagnostic method of the invention is itself devoid of any step applied to the body of the subject. Therefore, step a/of collecting information about the subject only comprises collecting the required information about the subject which is already available, and, optionally, if necessary, proceeding to measurements carried out on samples that have previously been isolated from the patient, in a conventional manner.

In particular embodiments of the invention, step a/comprises measuring the concentration of cytokeratin 18 or the fragment M65 or the fragment M30 thereof, and optionally the concentration of α-2-macroglobulin, in an isolated blood sample which has been obtained from the subject. To this end, any measurement method known by the person skilled in the art can be used.

In preferred embodiments of the invention, the blood sample has been collected from the subject less than ten days after cessation of alcohol consumption by said subject. It may have been collected either during a period of alcohol consumption, or after such a period, within the ten days following alcohol cessation. The diagnostic method of the invention has indeed been found to be less reliable when the blood concentration of cytokeratin 18, the fragment M65 or the fragment M30 thereof, is measured on blood samples collected more than ten days after the last alcohol consumption by the subject.

By way of example, the concentration of cytokeratin 18, the fragment M65 or the fragment M30 thereof, in the blood sample can be carried out using commercially available kits, such as, for example, the M65 EpiDeath® (Apoptosense®) ELISA kit for assaying the fragment M65 and the M30 Apoptosense® ELISA kit for assaying the fragment M30 of cytokeratin 18.

The concentration of α-2-macroglobulin in the blood sample of the subject can for example be measured using an immunoturbidimetry kit as provided by the company DiAgam, and a cobas c analyser system.

The body mass index of the subject can be obtained from values of weight and height given by the subject or measured on the subject, by dividing the weight of the subject by his height.

The data representative of liver stiffness can be as obtained by any ex vivo non-invasive imaging technique known to the person skilled in the art. By way of examples, this data can be obtained by magnetic resonance imaging (MRI), acoustic radiation force impulse imaging (ARFI), or by elastography, such as shear wave elastography (SWE) or two-dimensional shear wave elastography (2D-SWE), applied to the liver of the subject. In particularly preferred embodiments of the invention, the data representative of liver stiffness is as obtained by performing ex vivo transient elastography on the liver of the subject. Any conventional non-invasive transient elastography technique can be used for this purpose, such as vibration-controlled transient elastography, also known as FibroScan®.

The mathematical function used in the method of the invention is preferably a logistic multivariate function obtained by logistic regression. More particularly, it is a sigmoid function where the exponential term is a linear combination of several parameters, each of which is proportional to one of the variables described above.

The mathematical function used in the method of the invention has been obtained by evaluating, in particular by liver biopsy, the presence or absence of alcoholic steatohepatitis in a large cohort of patients, and dividing said patients into a first group of patients with alcoholic steatohepatitis and a second group of patients without alcoholic steatohepatitis, then identifying by univariate analysis the markers, in particular the biochemical markers, for which the values differ significantly between the two groups, and performing a logistic regression analysis to assess and ponder the independent discriminative values of the variables thus identified as being associated with the presence of alcoholic steatohepatitis, and obtaining a function combining these variables.

A mathematical function according to the invention, providing a probability of the occurrence of steatohepatitis, in particular alcoholic steatohepatitis, in a subject, having a high diagnosis performance, is represented by Equation (1):

$$P = 1 / ( 1 + Exp ( - ( a + b \times K 18 + c \times TE + d \times α2 M + e \times BMI + f \times age ) ) )$$

wherein
P is the probability of presence of the disease,
a, b, c, d, e, f are constant coefficients,
K18, TE, α2M, BMI and age are variables such that:
   K18 represents the concentration of cytokeratin 18 or the fragment M65 or the fragment M30 thereof, as measured in the blood sample obtained from the subject, preferably in IU/L,
   TE represents the data representative of liver stiffness, as measured ex vivo on the liver of the subject by a non-invasive imaging technique, preferably by transient elastography, preferably in kPa,
   α2M represents the concentration of α-2-macrogobulin as measured in the blood sample obtained by the subject, preferably in g/L,
   BMI represents the body mass index of the subject, preferably in kg/m²,
   age represents the age of the subject, preferably in years.

In particular embodiments of the invention, the coefficients a, b and f are such that, when the variables are expressed in the preferred units indicated above (if not, it is within the skills of the person skilled in the art to modify the values of the coefficients indicated below according to the units in which the variables are expressed):
−19.4≤a≤−8.4, preferably −14.5≤a≤−8.4, more preferably −14.5≤a≤−9.0 and even more preferably −14.5≤a≤−13.0,
and/or 0.001≤b≤0.002,
and/or 0.03≤f≤0.15, preferably 0.05≤f≤0.11, more preferably 0.06≤f≤0.11 and even more preferably 0.09≤f≤0.11.

Furthermore, in particular embodiments of the invention, when the variables are expressed in the preferable units indicated above:
c=0; and 0.51≤d≤2.38, preferably 1.30≤d≤1.80 and more preferably 1.33≤d≤1.37; and 0.04≤e≤0.28, preferably 0.10≤e≤0.17 and more preferably 0.14≤e≤0.17,
or: 0.04≤c≤0.17, preferably 0.06≤c≤0.11 and more preferably 0.09≤c≤0.11; and d=0; and 0.04≤e≤0.28, preferably 0.10≤e≤0.17 and more preferably 0.14≤e≤0.17,
or: 0.04≤c≤0.17, preferably 0.06≤c≤0.11 and more preferably 0.09≤c≤0.11; and 0.51≤d≤2.38, preferably 1.30≤d≤1.80 and more preferably 1.33≤d≤1.37; and e=0,
or: 0.04≤c≤0.17, preferably 0.06≤c≤0.11 and more preferably 0.09≤c≤0.11; and 0.51≤d≤2.38, preferably 1.30≤d≤1.80 and more preferably 1.33≤d≤1.37; and 0.04≤e≤0.28, preferably 0.10≤e≤0.17 and more preferably 0.14≤e≤0.17.

In preferred embodiments of the invention, in Equation (1), the coefficients a, b, c, d, e and f are such that, when the variables are expressed in the preferred units indicated above:
−14.5≤a≤−8.4, preferably −14.5≤a≤−9.1 and more preferably
14.5≤a≤−13.0,
0.001≤b≤0.002,
C=0 or 0.06≤c≤0.11 and preferably 0.09≤c≤0.11,
d=0 or 1.30≤d≤1.80 and preferably 1.33≤d≤1.37,
e=0 or 0.10≤e≤0.17 and preferably 0.14≤e≤0.17,
and 0.05≤f≤0.11, preferably 0.06≤f≤0.11 and more preferably 0.09≤f≤0.11,
   provided that c, d and e are not all simultaneously equal to 0.

In particularly preferred embodiments of the invention, in Equation (1), the coefficients a, b, c, d, e and f are such that, when the variables are expressed in the preferred units indicated above:

$$- 19.4 \leq a \leq - 9. , preferably - 14.5 \leq a \leq - 13. ,$$

$$0.001 \leq b \leq 0.002 ,$$

$$0.04 \leq c \leq 0.17 , preferably 0.09 \leq c \leq 0.11 ,$$

$$0.51 \leq d \leq 2.38 , preferably 1.33 \leq d \leq 1.37 ,$$

$$0.04 \leq e \leq 0.28 , preferably 0.14 \leq e \leq 0.17 ,$$

$$and 0.03 \leq f \leq 0.15 , preferably 0.05 \leq f \leq 0.11 .$$

More preferably, the mathematical function according to the invention is represented by Equation (2):

$$P = 1 / ( 1 + Exp ( - ( - {a}_{2} + {b}_{2} \times K 18 + {c}_{2} \times TE + {d}_{2} \times α2 M + {e}_{2} \times BMI + {f}_{2} \times age ) ) )$$

wherein K18, TE, α2M, BMI and age are as defined above and the coefficients a₂, b₂, c₂, d₂, e₂ and f₂ are such that, when the variables are expressed in the preferred units indicated above:

$$- 13.8 \leq {a}_{2} \leq - 13.7 , preferably - 13.72 \leq {a}_{2} \leq - 13.71 ,$$

$$0.001 \leq {b}_{2} \leq 0.002 , preferably {b}_{2} = 0.001 ,$$

$$0.1 \leq {c}_{2} \leq 0.11 ,$$

$$1.3 \leq {d}_{2} \leq 1.4 , preferably 1.35 \leq {d}_{2} \leq 1.36 ,$$

$$0.15 \leq {e}_{2} \leq 0.16 ,$$

$$and 0.08 \leq {f}_{2} \leq 0.1 , preferably 0.09 \leq {f}_{2} \leq 0.1 .$$

This mathematical function according to the invention demonstrates a particularly high accuracy, with an AUC of 0.93.

Then, in particular embodiments of the invention, the predetermined first reference value and the predetermined second reference value are such that:
the predetermined first reference value is substantially equal to 0.66 and the subject is considered as having steatohepatitis, in particular alcoholic steatohepatitis, if the score calculated by the method of the invention is higher than or equal to 0.66—this value being associated with a sensibility of the diagnostic method of 95%;
and/or the predetermined second reference value is substantially equal to 0.13 and the subject is considered as not having steatohepatitis, in particular alcoholic steatohepatitis, if the score is lower than or equal to 0.13—this value being associated with a specificity of the diagnostic method of 95%.

If the score is between 0.13 and 0.66, additional steps are to be taken in order to obtain a reliable diagnosis and/or a follow-up of the patient has to be planned, comprising another evaluation after a given lapse of time.

In a large, homogeneous population of heavy drinkers initiating an alcohol detoxification protocol, using Equation (2) above and such cut-off decision values to rule out (P≤0.13) or rule in (P≥0.66) alcoholic steatohepatitis, the score of the method of the invention is advantageously able to correctly classify a percentage as high as 65.3% of patients with a high level of accuracy.

Another mathematical function according to the invention, which does not include the data representative of liver stiffness, is represented by Equation (3):

$$P = 1 / ( 1 + Exp ( - ( - {a}_{3} + {b}_{3} \times K 18 + {d}_{3} \times α2 M + {e}_{3} \times BMI + {f}_{3} \times age ) ) )$$

wherein K18, α2M, BMI and age are as defined above and the coefficients a₃, b₃, d₃, e₃ and f₃ are such that, when the variables are expressed in the preferred units indicated above:

$$- 12. \leq {a}_{3} \leq - 11.9 , preferably - 11.97 \leq {a}_{1} \leq - 11.96 ,$$

$$0.001 \leq {b}_{3} \leq 0.002 , preferably {b}_{3} = 0.001 ,$$

$$1.7 \leq {d}_{3} \leq 1.8 , preferably 1.77 \leq {d}_{3} \leq 1.78 ,$$

$$0.12 \leq {e}_{3} \leq 0.13 ,$$

$$and 0.07 \leq {f}_{3} \leq 0.08 .$$

This mathematical function according to the invention demonstrates a very high accuracy, with an AUC higher than 0.89.

Another mathematical function according to the invention, which does not include the value of the concentration of α-2-macrogobulin, is represented by Equation (4):

$$P = 1 / ( 1 + Exp ( - ( - {a}_{4} + {b}_{4} \times K 18 + {c}_{4} \times TE + {e}_{4} \times BMI + {f}_{4} \times age ) ) )$$

wherein K18, TE, BMI and age are as defined above and the coefficients a₄, b₄, c₄, e₄ and f₄ are such that, when the variables are expressed in the preferred units indicated above:

$$- 8.5 \leq {a}_{4} \leq - 8.4 , preferably - 8.49 \leq {a}_{4} \leq - 8.48 ,$$

$$0.001 \leq {b}_{4} \leq 0.002 , preferably {b}_{4} = 0.001 ,$$

$$0.06 \leq {c}_{4} \leq 0.07 ,$$

$$0.1 \leq {e}_{4} \leq 0.11 ,$$

$$and 0.07 \leq {f}_{4} \leq 0.08 .$$

This mathematical function according to the invention demonstrates a very high accuracy, with an AUC of 0.90.

Another mathematical function according to the invention, which does not include the value of the body mass index, is represented by Equation (5):

$$P = 1 / ( 1 + Exp ( - ( - {a}_{5} + {b}_{5} \times K 18 + {c}_{5} \times TE + {d}_{5} \times α2 M + {f}_{5} \times age ) ) )$$

wherein K18, TE, α2M and age are as defined above and the coefficients a₅, b₅, c₅, d₅ and f₅ are such that, when the variables are expressed in the preferred units indicated above:

$$- 9.6 \leq {a}_{5} \leq - 9.5 , preferably - 9.52 \leq {a}_{5} \leq - 9.51 ,$$

$$0.001 \leq {b}_{5} \leq 0.002 , preferably {b}_{5} = 0.001 ,$$

$$0.09 \leq {c}_{5} \leq 0.1 ,$$

$$1.3 \leq {d}_{5} \leq 1.4 , preferably 1.35 \leq {d}_{5} \leq 1.31 ,$$

$$and 0.08 \leq {f}_{5} \leq 0.09 .$$

This mathematical function according to the invention demonstrates a very high accuracy, with an AUC higher than 0.91.

The invention also relates to a computer program product comprising a set of program code instructions which, when they are executed by a processor, implement step b/, optionally part of step a/ (in particular for the calculation of the body mass index from the values of the weight and the height of the subject), optionally step c/and preferentially also step d/, of the method of the invention, and more particularly, at least: receive as an input the values of the variables collected from a given subject, calculate the score by combining these values in a mathematical function according to the invention, and provide the score obtained as an output. It can also, optionally: compare this score to the predetermined first reference value, and optionally to the predetermined second reference value, and, optionally edit a statistical conclusion on whether the subject has steatohepatitis, more particularly alcoholic steatohepatitis, or, optionally, does not have steatohepatitis, more particularly alcoholic steatohepatitis, or should be subject to other medical examinations or to an additional subsequent monitoring, according to the result of the comparison(s) made.

A device for implementing these steps of the method of the invention can for example comprise at least one processor and at least one electronic memory wherein such a computer program product is stored.

### Management

A further object of the invention is an in vitro method of monitoring the evolution of steatohepatitis, more particularly alcoholic steatohepatitis, disease in a subject, which has previously been diagnosed as having steatohepatitis, more particularly alcoholic steatohepatitis.

This monitoring method comprises:
a/ at a first specific time of the disease, then at a second subsequent specific time of the disease, collecting for said subject: a value of the concentration of cytokeratin 18, the fragment M65 or the fragment M30 thereof, and optionally a value of the concentration of α-2-macroglobulin, in an isolated blood sample obtained from said subject; optionally, a data representative of liver stiffness as obtained by an ex vivo imaging technique, in particular by performing ex vivo transient elastography on the liver of said subject; and the value of the age, and optionally the value of the body mass index, of the subject—it is to be understood here that the actual operations having to be implemented at, respectively, the first specific time of the disease, then the second specific time of the disease, are the isolation of the blood sample from the subject, the ex vivo measurement of liver stiffness and the collection of height and weight of the subject, and that the other operations, in particular the blood concentration measurements, the gathering of all the information, the calculation of the body mass index, etc., can themselves be carried out at any time later on,
b/ for each of the first specific time of the disease and the second specific time of the disease: combining in a mathematical function the values thus collected of the age of the subject, the concentration in said blood sample of cytokeratin 18, the fragment M65 or the fragment M30 thereof, and at least two of: the concentration of α-2-macroglobulin in said blood sample, the data representative of liver stiffness and the value of the body mass index of the subject, in order to obtain a score, namely, respectively a first score and a second score,
c/ comparing the second score with the first score,
d/ and concluding that:
   the disease has evolved in a worse manner when the second score is higher than the first score,
   the disease has subsided, i.e., evolved towards healing, when the second score is lower than the first score,
   and the disease has not evolved when the second score is substantially equal to the first score.

The monitoring method of the invention proves particularly useful for monitoring the evolution of steatohepatitis, more particularly alcoholic steatohepatitis, at regular intervals subsequently to a change in the life habits of the subject, in particular after alcohol withdrawal or a diet change, or for monitoring the evolution of the disease in the absence of such changes.

The monitoring method of the invention, and each of the steps thereof, can have any feature or combination of features as described above referring to the method of the invention of diagnosing steatohepatitis in a subject.

In particular, the subject is preferably a mammal, more preferably a human.

The steatohepatitis is preferably alcoholic steatohepatitis. It can otherwise be non-alcoholic steatohepatitis (NASH).

The subject is preferably asymptomatic with respect to steatohepatitis, more particularly alcoholic steatohepatitis.

In particular embodiments of the invention, step a/ comprises measuring the concentration of cytokeratin 18, the fragment M65 or the fragment M30 thereof, and optionally the concentration of α-2-macroglobulin, in an isolated blood sample obtained from the subject.

The concentration of α-2-macroglobulin in the blood sample, the value of the body mass index of the subject and the data representative of liver stiffness are preferably all three included in the mathematical function. This function is preferably represented by Equation (1) above, for example by Equation (2) above.

Another aspect of the invention is an in vitro method of monitoring the effectiveness of a treatment of steatohepatitis, more particularly alcoholic steatohepatitis, in a subject which has previously been diagnosed as having steatohepatitis, more particularly alcoholic steatohepatitis. The treatment may be a curative treatment, comprising administering a drug to the subject, it may be bariatric surgery, and/or it may be the cessation of alcohol consumption and/or the change in the subject's diet. This monitoring method comprises:
a/ at a time before the start of the treatment, then at a specific time during the treatment or after the treatment, collecting for said subject: a value of the concentration of cytokeratin 18, the fragment M65 or the fragment M30 thereof, and optionally a value of the concentration of α-2-macroglobulin, in an isolated blood sample obtained from the subject; optionally, a data representative of liver stiffness as obtained by an ex vivo imaging technique, in particular by performing ex vivo transient elastography on the liver of said subject; and the value of the age, and optionally the value of the body mass index, of the subject—it is to be understood here that the actual operations having to be implemented at, respectively, the time before the start of the treatment, then the specific time during the treatment or after the treatment, are the isolation of the blood sample from the subject, the ex vivo measurement of liver stiffness and the collection of height and weight of the subject, and that the other operations, in particular the blood concentration measurements, the gathering of all the information, the calculation of the body mass index, etc., can themselves be carried out at any time later on,
b/ for each of the time before the start of treatment and the specific time during or after the treatment: combining in a mathematical function the values thus collected of the age of the subject, the concentration in said blood sample of cytokeratin 18, the fragment M65 or the fragment M30 thereof, and at least two of: the concentration of α-2-macroglobulin in said blood sample, the data representative of liver stiffness and the value of the body mass index of the subject,
in order to obtain a score,
c/ comparing the score obtained for the specific time during or after the treatment with the score obtained for the time before the start of the treatment,
d/ and concluding that:
   the treatment is efficient when the score obtained for the specific time during or after the treatment is lower than the score obtained for the time before the start of the treatment,
   the treatment is inefficient, or not efficient enough, when the score obtained for the specific time during or after the treatment is higher than or equal to the score obtained for the time before the start of treatment.

The result of such a monitoring method proves very useful to the practitioner for deciding on the continuation of the treatment, or the replacement or complementation thereof with a different treatment.

This monitoring method, and each of the steps it comprises, can have any feature or combination of features as described above referring to the method of the invention of diagnosing steatohepatitis in a subject.

In particular, the subject is preferably a mammal, more preferably a human.

The steatohepatitis is preferably alcoholic steatohepatitis. It can otherwise be non-alcoholic steatohepatitis (NASH).

The subject is preferably asymptomatic with respect to steatohepatitis, more particularly alcoholic steatohepatitis.

In particular embodiments of the invention, step a/comprises measuring the concentration of cytokeratin 18, the fragment M65 or the fragment M30 thereof, and optionally the concentration of α-2-macroglobulin, in an isolated blood sample obtained from the subject.

The concentration of α-2-macroglobulin in the blood sample, the value of the body mass index of the subject and the data representative of liver stiffness are preferably all three included in the mathematical function. This function is preferably represented by Equation (1) above, for example by Equation (2) above.

### Therapeutic Method

An additional object of the invention is a method of treating steatohepatitis, more particularly alcoholic steatohepatitis, with an appropriate treatment in a subject wherein steatohepatitis, more particularly alcoholic steatohepatitis, has been positively diagnosed by an in vitro method of diagnosing steatohepatitis according to the invention.

In particular, this treatment method is carried out when, in the embodiments of the invention wherein the mathematical function used in the diagnostic method of the invention is represented by Equation (2) above, the calculated score is higher than or equal to 0.66.

As indicated above, the appropriate treatment can be the cessation of alcohol consumption and/or a suitable healthier dietary treatment. It can also or otherwise comprise the administration to said subject of a suitable drug, such as vitamin E, pioglitazone, liraglutide, semaglutide, obeticholic acid, elafibranor, cenicriviroc, armachol, resmetirom, or any other of the molecules cited in the publication of Rowe et al., 2022. It can also be bariatric surgery.

“Treating” herein means reversing, alleviating, inhibiting the progress of, or preventing, the disorder or condition to which such term applies, or reversing, alleviating inhibiting the progress of, or preventing one or more symptoms of the disorder or condition to which such term applies.

Yet another aspect of the invention is a method of treating steatohepatitis, more particularly alcoholic steatohepatitis, in a subject, which method comprises:
a/ collecting for said subject: a value of the concentration of cytokeratin 18, the fragment M65 or the fragment M30 thereof, and optionally a value of the concentration of α-2-macroglobulin, in an isolated blood sample obtained from the subject; optionally, a data representative of liver stiffness as obtained by an ex vivo imaging technique, in particular by performing ex vivo transient elastography on the liver of the subject; the value of the age, and optionally the value of the body mass index, of the subject,
b/ combining in a mathematical function the values thus collected of the age of the subject, the concentration in said blood sample of cytokeratin 18, the fragment M65 or the fragment M30 thereof, and at least two of: the concentration of α-2-macroglobulin in said blood sample, the data representative of liver stiffness and the value of the body mass index of the subject, in order to obtain a score,
c/ comparing this score to a predetermined first reference value,
d/ and if said score is higher than or equal to this first reference value, concluding that the subject has steatohepatitis, more particularly alcoholic steatohepatitis, and treating the subject with an appropriate treatment.

In particular, the therapeutic method of the invention can comprise a step of administering to the subject in need thereof, i.e., which has been diagnosed as having steatohepatitis, more particularly alcoholic steatohepatitis, in step d/of the method, a therapeutically effective amount of an appropriate drug for treating steatohepatitis, more particularly alcoholic steatohepatitis, or at least a symptom thereof, such as the molecules cited above.

It can alternatively, or in combination, comprise a non-medicated management of the disease, such as cessation of alcohol consumption and/or an appropriate diet.

It can also consist of, or comprise, bariatric surgery.

This treatment method, and each of the steps it comprises, can have any feature or combination of features as described above referring to the method of the invention of diagnosing steatohepatitis in a subject.

In particular, the subject is preferably a mammal, more preferably a human.

The steatohepatitis is preferably alcoholic steatohepatitis. It can otherwise be non-alcoholic steatohepatitis (NASH).

The subject is preferably asymptomatic with respect to steatohepatitis, more particularly alcoholic steatohepatitis.

In particular embodiments of the invention, step a/comprises measuring the concentration of cytokeratin 18, the fragment M65 or the fragment M30 thereof, and optionally the concentration of α-2-macroglobulin, in an isolated blood sample obtained from the subject.

The concentration of α-2-macroglobulin in the blood sample, the value of the body mass index of the subject and the data representative of liver stiffness are preferably all three included in the mathematical function. This function is preferably represented by Equation (1) above, for example by Equation (2) above.

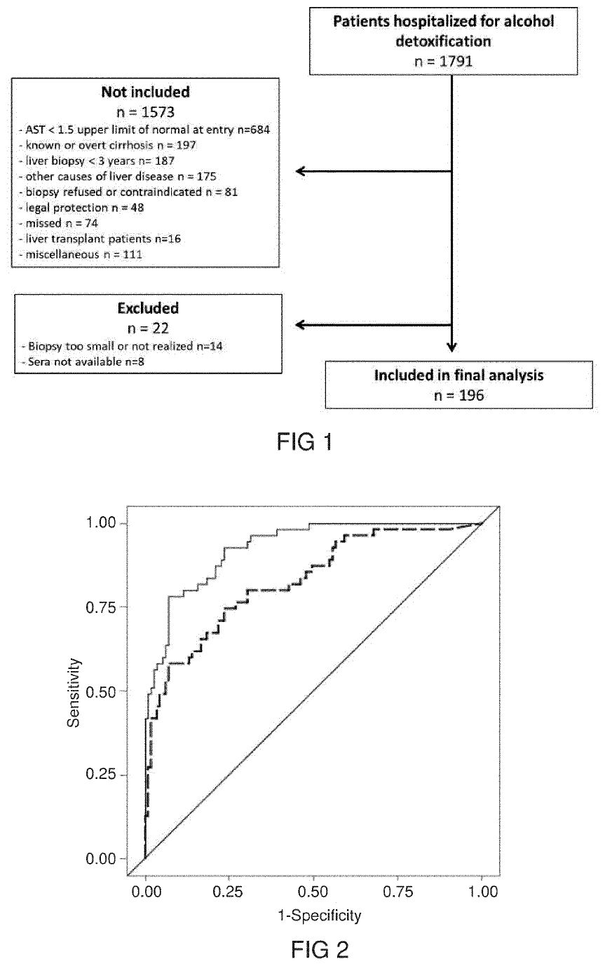

The features and advantages of the invention will emerge more clearly in the light of the following examples, provided for illustrative purposes only and in no way limitative of the scope of the invention, with the support of FIGS. 1 to 8, in which:

FIG. 1 represents a flow chart of patient inclusion in a test cohort studied for the assessment of the multivariate model for alcoholic steatohepatitis diagnostic of the invention;

FIG. 2 represents the ROC curves for the diagnostic of alcoholic steatohepatitis in the studied test population for a univariate model including the K18-M65 fragment alone (dotted line) and for a multivariate model according the invention including 5 variables (full line);

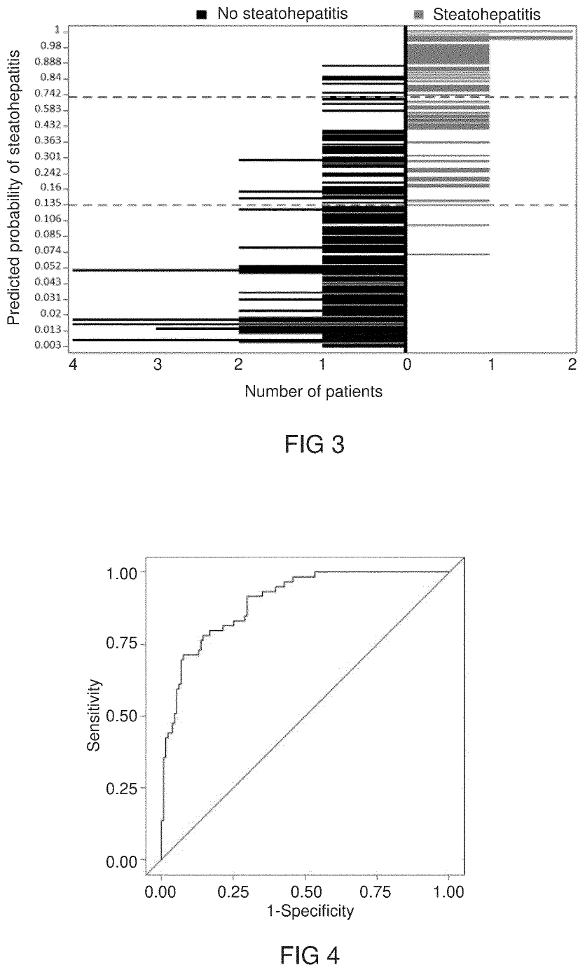

FIG. 3 represents a graph showing the predicted probability of alcoholic steatohepatitis according to histological findings, as obtained for a multivariate model according the invention including 5 variables. The dotted lines represent the thresholds for ruling out (P≤0.135) for a sensibility ≥95% or ruling in (P≥0.667) for a specificity ≥95% alcoholic steatohepatitis in the studied test population of heavy drinkers who were asymptomatic patients hospitalized for alcohol withdrawal;

FIG. 4 represents the ROC curve for the diagnostic of alcoholic steatohepatitis in the studied test population for a model according to the invention including 4 parameters (excluding α-2-macroglobulin);

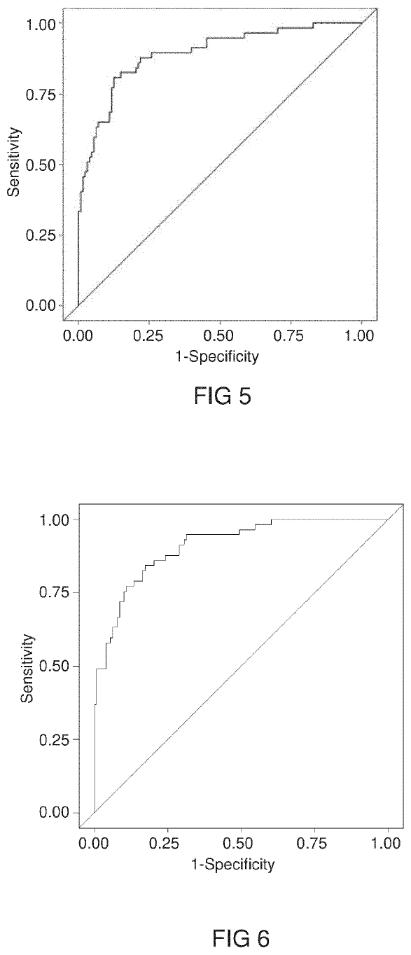

FIG. 5 represents the ROC curve for the diagnostic of alcoholic steatohepatitis in the studied test population for a model according to the invention including 4 parameters (excluding result of transient elastography);

FIG. 6 represents the ROC curve for the diagnostic of alcoholic steatohepatitis in the studied test population for a model according to the invention including 4 parameters (excluding body mass index);

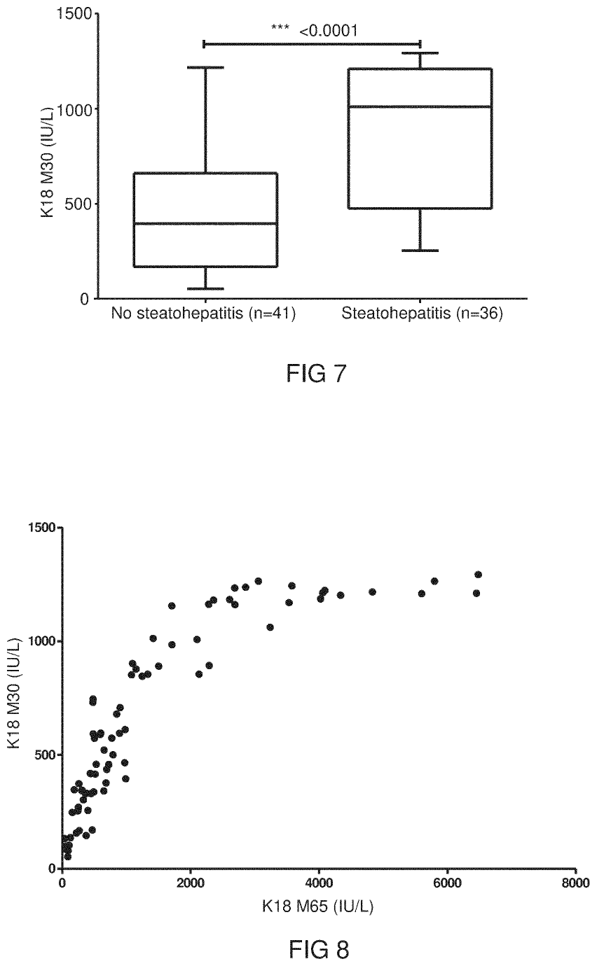

FIG. 7 shows a graph representing the degree of significance of the mean serum concentration of the fragment M30 of cytokeratin 18 (K18) between patients without alcoholic steatohepatitis (n=41 patients) or with alcoholic steatohepatitis (n=36 patients) as obtained using a Mann-Whitney test with α=0.05;

FIG. 8 shows a graph representing the degree of correlation between the serum concentration of the fragment M65 of cytokeratin 18 (K18) and the serum concentration of the fragment M30 of cytokeratin 18 (K18) in a test cohort of patients with or without alcoholic steatohepatitis, as obtained using the Spearman method with «=0.05.

### Example 1—Study on a Test Cohort of Patients

### Experimental Procedures

### Patients

A total of 204 patients were considered for the study. The patients were recruited at Pontchaillou University Hospital as part of a multicenter clinical study (clinical trial number NCT01789008) aiming to evaluate the diagnostic value of TE of liver fibrosis. Written consent was obtained from each patient. Consecutive patients hospitalized for alcohol detoxification were screened. Inclusion criteria at the hospital admission were aspartate aminotransferase (AST)>1.5 the upper limit of normal (35 IU/L), elevated gamma-glutamyl-transferase (γGT)>50 IU/L, age >18 years, and excessive alcohol consumption (>30 g/day or 20 g/day for men or women) for at least 5 years. When patients were recruited, overt or known cirrhosis and other causes of liver disease led to study exclusion. However, based on biological results, metabolic cofactors were acceptable when alcohol was the major cause of liver disease. Transient Elastography (TE) and liver biopsy were performed the same day as venous blood collection, within 10 days after cessation of alcohol consumption.

### Assessment of Steatohepatitis and Fibrosis by Liver Histology

Biopsies ≥10 mm were considered. Each liver biopsy was read by two blinded and experienced liver pathologists from two different centers (BT and SM). In the case of discrepancy regarding fibrosis stage, both pathologists met to reach a consensus. Liver fibrosis was staged according to METAVIR score: F0, no fibrosis; F1, portal fibrosis; F2, mild extensive portal fibrosis (less than half of portal tracks have septa); F3, severe extensive portal fibrosis; and F4, cirrhosis. Alcoholic steatohepatitis was defined by the presence of steatosis in >5% of hepatocytes, hepatocellular ballooning of any degree, and any amount of lobular inflammatory infiltrates (Bedossa et al., 2012).

### Transient Elastography

Patients underwent TE (Echosens®, Paris, France) after overnight fasting. TE was considered valid if at least 10 measures were performed and if the interquartile range (IQR) was <30% of the median or if the median was <7.1 kPa.

### Baseline Demographic, Biochemistry, and Other Biological Data

Age, gender, and body mass index (BMI) were recorded at inclusion. Data regarding alcohol consumption were recorded using a self-questionnaire. AST, alanine aminotransferase (ALT), γGT, platelets, iron, ferritin and hyaluronic acid (HA) were measured. Bilirubin was measured in frozen (−80° C.) sera taken the day of the biopsy.

### Circulating K18-M65 Fragment and α-2-Macroglobulin (α2M) Detection

The blood sample was collected the same day than TE and liver biopsy, centrifuged, aliquoted, and kept frozen at −80° C. until used by the Centre de Ressources Biologiques Santé of Rennes BB-0033-00056.

Serum concentrations of K18-M65 fragments were measured using the M65 EpiDeath® (Apoptosense) ELISA kit (Peviva-VLVbio, Sweden) following the supplier's protocol.

Serum concentrations of α2M were measured using an immunoturbidimetry kit as provided by DiAgam and a cobas c analyser system, following the supplier's protocol.

### Statistical Analysis

Logistic regression models were used to estimate the association between alcoholic steatohepatitis (presence/absence), K18-M65 blood levels, and other risk factors in univariate analyses. To adjust for various covariates, a multivariate logistic model was used with mixed (forward/backward) stepwise selection including all variables associated with alcoholic steatohepatitis in univariate analyses (p<0.20). The accuracy of K18-M65 blood levels for the diagnostic of alcoholic steatohepatitis was evaluated by analyzing the area under the receiver operating characteristics curve (AUC). The sensitivity, specificity, PPV, NPV, and 95% confidence interval (CI) were calculated for various cut-offs of K18-M65 levels, optimizing either the specificity (≥95%) or sensitivity (>95%) or based on the Youden index. For sensitivity analyses, multivariate regression analyses were performed excluding either TE (to rely only on demographic or biological variables) or α2M, which is less commonly measured in daily clinical practice.

Results are expressed as odds ratios (ORs) with 95% CIs. Internal validation of the diagnostic models was performed with resampling method and calibration test. A 1000-fold bootstrap resampling including the previously selected predictors was used to obtain optimism-corrected AUC (cAUC). The calibration of the models was tested with Hosmer-Lemeshow goodness-of-fit. The closer the predicted probabilities to the observed probabilities with p-values of Hosmer-Lemeshow goodness-of-fit test (p_{HL}) greater than 0.05, the better the model is calibrated.

P-values <0.05 were considered significant, and all tests were two-sided. All analyses were performed using SAS® version 9.4 statistical software and R software, version 4.1.2 (rms package).

### Results

### Demographic Characteristics and Biological Data

The inclusion period lasted from February 2013 to April 2017. The flow chart of patient inclusion is presented in FIG. 1. Frozen sera were available for 196 patients, who then constituted the study population. Histological findings of steatohepatitis were found for 61 (31%) patients in the liver biopsy. The main demographic characteristics and biological data are shown in Table 1 for the two groups.

TABLE 1
Baseline demographic characteristics, biological data, alcohol consumption,
fibrosis stage and transient elastography results among patients with or without
histological findings of alcoholic steatohepatitis from liver biopsy.

| | Absence of | | Presence of | | Missing |
|---|---|---|---|---|---|
| | steatohepatitis | | steatohepatitis | | data |
| No. cases | 135 | | 61 | | — |
| Age (y) | 45 | [45-51] | 49 | [46-56] | 0 |
| Sex, male/female | 112/22 | | 41/20 | | 0 |
| BMI (kg/m²) | 22.4 | [17.1-39.1] | 24.0 | [16.0-37.8] | 3 |
| Duration of excessive drinking | 15 | [10-24] | 13 | [8-25] | 1 |
| (y) | | | | | |
| Daily alcohol drinking in the | 126 | [70-181] | 98 | [56-140] | 5 |
| month before hospitalization (g) | | | | | |
| Delay between last alcohol | 5.5 | [4.0-7.0] | 6.0 | [5.0-8.5] | 7 |
| consumption and transient | | | | | |
| elastography (d) | | | | | |
| Fibrosis stage n (%) | | | | | 0 |
| F0 | 16 | (11.9) | 0 | (0) | — |
| F1 | 89 | (65.9) | 20 | (32.8) | — |
| F2 | 21 | (15.6) | 16 | (26.2) | — |
| F3 | 3 | (2.2) | 16 | (26.2) | — |
| F4 | 6 | (4.4) | 9 | (14.8) | — |
| ALT (IU/I) | 70 | [44-109] | 67 | [40-109] | 0 |
| AST (IU/I) | 62 | [44-98] | 86 | [60-118] | 2 |
| γ-GT (IU/I) | 235 | [124-438] | 438 | [256-719] | 0 |
| Iron (μmol/L) | 14.3 | [10.9-19.7] | 13.0 | [9.6-16.9] | 3 |
| Ferritin (μg/L) | 451 | [254-695] | 752 | [277-1132] | 3 |
| Bilirubin (μmol/L) | 6.8 | [4.7-9.35] | 9.2 | [5.7-13.8] | 6 |
| α-2 macroglobulin (g/L) | 1.6 | [1.4-1.8] | 1.8 | [1.6-2.4] | 8 |
| Platelets (Giga/L) | 180 | [146-232] | 183 | [127-220] | 8 |
| Transferrin saturation (%) | 27.1 | [19.4-36.5] | 26.6 | [17.4-37.3] | 4 |
| Hyaluronic acid (μg/L) | 34 | [19-62] | 133 | [42-284] | 4 |
| Transient elastography (kPa) | 5.1 | [4.1-6.8] | 11.8 | [7.8-29.0] | 4 |

Results are expressed as median [interquartile range] unless otherwise noted.
ALT, alanine aminotransferase; AST, aspartate aminotransferase; BMI, body mass index; γ-GT, gamma-glutamyltransferase.

### Circulating K18-M65 Accurately Detects ASH

The mean circulating level of K18-M65 was significantly higher in patients diagnosed with alcoholic steatohepatitis (2249 IU/L) than the non-alcoholic steatohepatitis group (595 IU/L). Therefore, the diagnostic accuracy of this biomarker was evaluated and an AUC of 0.82 [95% CIs, 0.76-0.89] was reached (cAUC=0.82, p_{HL}=0.30) (FIG. 2, dotted line). The PPV and NPV were calculated for various cut-offs with either high specificity (≥95%) or high sensitivity (≥95%), or based on the highest Youden index. The results are shown in Table 2.

TABLE 2
Diagnostic accuracy of circulating K18-M65 levels for alcoholic
steatohepatitis in a population of heavy drinkers.

| K18-M65 cut-off | Sensitivity | Specificity | PPV | NPV |
|---|---|---|---|---|
| value (IU/L) | (%) | (%) | (%) | (%) |
| 323 | 95.1 | 42.2 | 42.6 | 95.0 |
| 2133 | 42.6 | 95.6 | 81.2 | 78.7 |
| 792 (Youden index) | 73.8 | 76.3 | 58.4 | 86.5 |
| 641* | 78.7 | 68.9 | 53.3 | 87.7 |
| 2000* | 45.9 | 94.1 | 77.8 | 79.4 |

PPV, positive predictive value; NPV, negative predictive value.
Various cut-offs aimed at optimizing specificity (>95%) or sensitivity (>95%) or were based on the Youden index.
*The diagnostic performance of previously published cut-offs is given as a reference (Wong et al., 2018)

Using these cut-offs, K18-M65 was able to accurately rule out a diagnostic of alcoholic steatohepatitis for 60 patients, covering 44.4% of the negative patients found on liver biopsy, with 3 false negative cases. For the diagnostic of alcoholic steatohepatitis, the circulating level of K18-M65 was able to rule in 32 patients, covering 52.5% of all positive patients, with 6 false positive cases. Altogether, the level of circulating K18-M65 was able to classify 92 (46.9%) of all patients.

Use of the Youden index resulted in an intermediate cut-off with poor diagnostic performance as opposed to the use of two thresholds. The diagnostic performance of cut-offs that were previously defined from studies on severe AH were also calculated and similar, albeit less precise, results were found (Table 2).

### High Diagnostic Performance of K18-M65-Based Multi-Parameter Scores

The initial univariate logistic regression considered all the biological and baseline demographic variables. Five covariates (TE, α2M, BMI, and age in addition to K18-M65 which was the first to enter the model) were selected in the multivariate analyses. The odds ratios, 95% confidence intervals and p-values for each of the covariates introduced into the multivariate logistic models are indicated in Table 3.

TABLE 3

| | Odds ratio | |
|---|---|---|
| Parameter | [95% confidence interval] | P-value |
| Transient elastography (kPa) | 1.106 [1.042-1.173] | <0.001 |
| Alpha-2-macroglobulin (g/L) | 3.871 [1.534-9.772] | 0.01 |
| Cytokeratin 18-M65 (IU/L) | 1.101 [1.001-1.002] | <0.0001 |
| Body mass index (kg/m²) | 1.169 [1.044-1.309] | <0.01 |
| Age (years) | 1.094 [1.037-1.154] | 0.001 |

Because of missing covariate data, only 170 patients of which 55 are diagnosed with alcoholic steatohepatitis were considered.

### 5-Variables Multivariate Model

This mathematical function corresponds to Equation (2) as defined above.

This multivariate model permitted an AUC of 0.93 [95% CI, 0.89-0.97] (cAUC=0.92, p_{HL}=0.60) to be reached (FIG. 2, full line). For predicted probability values ranging from 0 to 0.135, the model allows accurate classification of 79 (68.7%) of the non-steatohepatitis patients with 2 false negative cases, reaching a NPV of 97.3%. On the other hand, from 0.667 and above, the model can classify 32 (58.2%) of the patients with alcoholic steatohepatitis with 5 false positive cases (FIG. 3) with 86.5% PPV. Thus, this model with two cut-off decision points was able to accurately classify 111 (65.3%) of the patients considered in the analysis, about 40% more than K18-M65 alone.

### 4-Variables Multivariate Models

Three additional analyses were performed based on 4 variables according to the invention:
one without α2M, using the mathematical function represented by Equation (4) above,
one with only biological and demographic variables (without TE), using the mathematical function represented by Equation (3) above,
and one without BMI, using the mathematical function represented by Equation (5) above.

The results are presented in FIGS. 4, 5 and 6, respectively. The AUC was respectively of:
0.90 [95% CI, 0.86-0.94] (cAUC=0.90, p_{HL}=0.46) (without α2M),
0.89 [95% CI, 0.84-0.94] (cAUC=0.89, p_{HL}=0.90) (without TE)
0.91 (without BMI).

These results demonstrate a high performance of the methods of the invention using a 4-variables function, this performance being slightly lower than that of the method of the invention using the 5-variables mathematical function.

### Example 2—Comparison of Fragments M65 and M30 of K18

### Experimental Procedures

This experiment was performed on a reduced cohort of 77 patients hospitalized for alcohol detoxification (41 without steatohepatitis and 36 with alcoholic steatohepatitis).

Serum concentrations of K18-M65 and K18-M30 fragments were measured using the M65 or M30 EpiDeath® (Apoptosense®) ELISA kit (Peviva-VLVbio, Sweden) following the supplier's protocol. Both fragments were assayed on sera collected from the patients on the same day than TE and liver biopsy were realized, centrifuged, aliquoted, and kept frozen at −80° C. until used by the Centre de Ressources Biologiques Santé of Rennes BB-0033-00056.

The degree of significance of mean K18 M30 level between patients without or with steatohepatitis was assessed using a Mann-Whitney test with α=0.05.

The degree of correlation between K18 M65 and K18 M30 was assayed using Spearman method with α=0.05.

### Results

The degree of significance of the mean serum concentration of the fragment M30 of K18 between the patients with alcoholic steatohepatitis and the patients without alcoholic steatohepatitis is shown in FIG. 7. A highly significant difference (p<0.0001) is observed.

The degree of correlation of the serum concentration of the fragment M30 of K18 (K18 M30) and that of the fragment M65 of K18 (K18 M65) is shown in FIG. 8.

This graph shows a linear relationship between K18 M65 and K18 M30 for values of K18 M30 up to 1000 IU/L and then a plateau is reached. This is explained by a technical limitation of the K18-M30 ELISA kit which is unable to quantify above 1000 IU/L. Despite this limitation, the correlation is strong with Spearman R=0.9485 associated to a strong level of significance (p-value<0.0001).

These results demonstrate that fragments M30 and M65 of K18 can equivalently be used for the diagnosis of alcoholic steatohepatitis in the method of the invention.

### Example 3—Validation in an Independent Validation Cohort of 58 Patients

The results from the test cohort, described in Example 1, were validated in a pre-existing prospective cohort of heavy drinker patients hospitalized for alcohol detoxification at Salem Medical Center Heidelberg (Germany). Patients had TE and venous blood collection at the first day after hospitalization and liver biopsy soon after, mostly within 10 days.

The methods implemented were the same as described above in Example 1, except for the differences below.

Liver biopsy (mean biopsy lengths 15.6 mm, diameter 1.4 mm) was obtained by Menghini technique under ultrasound guidance. For histological analysis, the specimen was fixed in formalin and embedded in paraffin. Two experienced pathologists blinded to the patient's data analyzed all liver biopsies independently. 4 μm sections were dewaxed and stained with scoring of as well as fibrosis.

For transient elastography, both M and XL probes were used.

Among all patients of the validation cohort, only 58 patients had all required data available and constitute the validation cohort, whom 51 (88%) had histological findings of alcoholic steatohepatitis. The main demographic characteristics and biological data are shown in Table 4.

TABLE 4
Baseline demographic characteristics, biological data, alcohol consumption,
fibrosis stage and transient elastography results among patients with or
without histological findings of steatohepatitis from liver biopsy.

| | Absence of | | Presence of | | Missing |
|---|---|---|---|---|---|
| | steatohepatitis | | steatohepatitis | | data |
| No. cases | 7 | | 51 | | — |
| Age (y) | 50 | [42-51] | 55 | [49-64] | 0 |
| Sex, male/female | 5/2 | | 32/19 | | 0 |
| BMI (kg/m²) | 26.3 | [25.3-27.1] | 24.4 | [21.9-29.7] | 0 |
| Duration of excessive drinking (y) | 20 | [16-20] | 12 | [9-24] | 30 |
| Daily alcohol drinking in the month | 246 | [192-283] | 130 | [78-186] | 5 |
| before hospitalization (g) | | | | | |
| Delay between last alcohol | 0.5 | [0.0-1.0] | 0.0 | [0.0-1.0] | 13 |
| consumption and transient | | | | | |
| elastography (d) | | | | | |
| Fibrosis stage n (%) | | | | | 0 |
| F0 | 4 | (57.1) | 1 | (2.0) | — |
| F1-F3 | 3 | (42.9) | 25 | (49.0) | — |
| F4 | 0 | (0) | 25 | (49.0) | — |
| ALT (IU/I) | 47 | [35-83] | 56 | [35-88] | 0 |
| AST (IU/I) | 38 | [33-115] | 92 | [70-175] | 0 |
| γ-GT (IU/I) | 89 | [76-177] | 527 | [232-899] | 0 |
| Iron (μmol/L) | 15.6 | [10.2-36.3] | 16.1 | [12.2-24.9] | 5 |
| Ferritin (μg/L) | 114 | [80-407] | 737 | [187-1461] | 4 |
| Bilirubin (μmol/L) | 6.8 | [5.1-13.7] | 22.2 | [13.7-47.9] | 0 |
| α-2 macroglobulin (g/L) | 1.6 | [1.2-2.0] | 2.5 | [1.9-3.4] | 0 |
| Platelets (Giga/L) | 292 | [169-310] | 149 | [92-199] | 0 |
| Transferrin saturation (%) | 18 | [10-87] | 36.0 | [27.0-70.7] | 23 |
| Transient elastography (kPa) | 4.9 | [3.2-6.5] | 36.0 | [27.0-70.7] | 0 |

Results are expressed as median [interquartile range] unless otherwise noted.
ALT, alanine aminotransferase; AST, aspartate aminotransferase; BMI, body mass index; γ-GT, gamma-glutamyltransferase.

The best multivariate model (5 covariates, “full algorithm”) described in Example 1 was tested on the validation cohort and permitted to reach an AUC of 0.94 [95% ICs, 0.88-1.00], while K18-M65 alone provided an AUC of 0.90 [95% ICs, 0.80-1.00], as demonstrated by the results shown in table 5:

TABLE 5
Diagnostic performance of K18-M65 and the calculated predicted probability
from the best defined multiparametric algorithm for the presence of
biopsy-confirmed alcohol steatohepatitis in the validation cohort.

| | K18-M65 (IU/L) | | | | Full algorithm | | | |
|---|---|---|---|---|---|---|---|---|
| Cutoff | 323 | | 2133 | | 0.135 | | 0.667 | |
| Sensitivity (%) | 98.0 | [89.6-99.9] | 33.3 | [20.8-47.9] | 92.7 | [81.1-97.8] | 82.3 | [69.1-91.6] |
| Specificity (%) | 42.9 | [9.9-81.6] | 100.0 | [59.0-100.0] | 85.7 | [42.1-99.6] | 100.0 | [59.0-100.0] |
| PPV (%) | 92.6 | [86.8-96.0] | 100.0 | [81.6-100.0] | 97.9 | [88.4-99.7] | 100.0 | [91.6-100.0] |
| NPV (%) | 75.0 | [26.5-96.2] | 17.1 | [14.5-20.0] | 60.0 | [35.8-80.1] | 43.8 | [30.1-58.5] |

| | | |
|---|---|---|
| AUC | 0.90 [0.80-1.00] | 0.94 [0.88-1.00] |

PPV, positive predictive value; NPV, negative predictive value; AUC, Area Under the Curve Using the cut-offs defined in the test cohort, 5 out of 7 non-ASH patients were correctly classified, the 2 last remaining in the grey zone.
Thus, no false positive was seen.
On the opposite, 42 (82.4%) of the 51 ASH patients were correctly classified, 5 (9.8%) were found in the grey zone and 4 (7.8%) classified as false negatives.
All the 4 false negatives cases were classified with the lowest level of lobular inflammation from liver biopsy.
Altogether the best model with two cut-off decision points was able to accurately classify 47 (81.0%) of the patients from the validation cohort.

These results confirm the very high diagnostic accuracy for ASH of the method of the invention.

### REFERENCES

Bedossa et al., 2012. Hepatology. 56 (5): 1751-9
Bellentani et al., 2001. J Hepatol. 35 (4): 531-7
Chedid et al., 1991. Am J Gastroenterol. 86 (2): 210-6
Chrostek L et al., 2014. World J Gastroenterol. 20 (25): 8018-23
de Franchis et al., 2022. J Hepatol. 76 (4): 959-74
European Association for the Study of the Liver, 2018. J Hepatol. 69 (1): 154-81
Eyles et al., 2013. Br J Gen Pract. 63 (613): e516-22
Lackner et al., 2017. J Hepatol. 66 (3): 610-8
Greuter et al., 2017. JCI Insight. 2 (17)
Harrison et al., 2020. Lancet Gastroenterol Hepatol. 5 (11): 970-85
Leers et al., 1999. J Pathol. 187 (5): 567-72
Lucey, 2009. Clin Liver Dis. 13 (2): 267-75
Mathurin et al., 2007. Aliment Pharmacol Ther. 25 (9): 1047-54
Moreno et al., 2019. J Hepatol. 70 (2): 273-83
Piccinino et al., 1986. J Hepatol. 2 (2): 165-73
Romano et al., 1986. Differentiation. 30 (3): 244-53
Rowe et al., 2022. Clin Gastroenterol Hepatol. 20 (6): 1209-1217
Rudler et al., 2015. PLOS One. 10 (8): e0134302
Seitz et al., 2018. Nat Rev Dis Primers. 4 (1): 16
Stumptner et al., 2000. Am J Pathol. 156 (1): 77-90
Wong et al., 2018. Nat Rev Gastroenterol Hepatol. 15 (8): 461-78

1. An in vitro method of diagnosing alcoholic steatohepatitis in a subject, and treating the subject comprising:
a) collecting for said subject:
a value of a concentration of cytokeratin 18, a fragment M65 or a fragment M30 thereof, and optionally a value of a concentration of α-2-macroglobulin, in an isolated blood sample obtained from said subject,
optionally, a data representative of liver stiffness as obtained by an ex vivo imaging technique,
a value of age, and optionally of a body mass index,
b) combining in a mathematical function the value of the age of the subject, the concentration in said blood sample of cytokeratin 18, the fragment M65 or the fragment M30 thereof, and at least two of: the concentration of α-2-macroglobulin in said blood sample, the data representative of liver stiffness and the value of the body mass index of the subject, in order to obtain a score,
c) determining that the score is higher than or equal to a predetermined first reference value, and
d) treating the subject determined to have a score that is higher than or equal to said first reference value with a treatment that is suitable for alcoholic steatohepatitis.

2. The method according to claim 1, wherein subject is a human.

3. The method according to claim 1, wherein subject is asymptomatic.

4. The method according to claim 1, comprising comparing said score with a predetermined second reference value and concluding that the subject does not have alcoholic steatohepatitis when said score is lower than or equal to said second reference value.

5. The method according to claim 1, wherein
the concentration of α-2-macroglobulin in said blood sample, the data representative of liver stiffness and the value of the body mass index of the subject are all included in said mathematical function.

6. The method according to claim 1, wherein step a) comprises measuring the concentration of cytokeratin 18, the fragment M65 or the fragment M30 thereof, and optionally the concentration of α-2-macroglobulin, in an isolated blood sample obtained from said subject.

7. The method according to claim 1, wherein said blood sample is collected from said subject less than ten days after cessation of alcohol consumption by said subject.

8. The method according to claim 1, wherein said mathematical function is a logistic multivariate function obtained by logistic regression.

9. An in vitro method of monitoring alcoholic steatohepatitis in a subject having the disease, and treating the subject, comprising:
a) at a first specific time of the disease then at a second specific time of the disease, collecting for said subject:
a value of a concentration of cytokeratin 18, a fragment M65 or a fragment M30 thereof, and optionally a value of a concentration of α-2-macroglobulin, in an isolated blood sample obtained from said subject;
optionally, a data representative of liver stiffness as obtained by an ex vivo imaging technique; and
a value of the age, and optionally of a body mass index, of the subject,
b) for each of the first specific time of the disease and the second specific time of the disease, combining in a mathematical function the value of the age of the subject, the concentration in said blood sample of cytokeratin 18, the fragment M65 or the fragment M30 thereof, and at least two of: the concentration of α-2-macroglobulin in said blood sample, the data representative of liver stiffness and the value of the body mass index of the subject, in order to obtain a score,
c) treating the subject having a score obtained for the second specific time of the disease that is higher than the score obtained for the first specific time of the disease with a treatment suitable for a subject whose disease has worsened.

10. (canceled)

11. (canceled)

12. A method of treating alcoholic steatohepatitis in a subject, comprising:
a) collecting for said subject: a value of the concentration of cytokeratin 18, a fragment M65 or a fragment M30 thereof, and optionally a value of a concentration of α-2-macroglobulin, in an isolated blood sample obtained from said subject; and optionally, a data representative of liver stiffness obtained by an ex vivo imaging technique; a value of age, and optionally of a body mass index, of the subject,
b) combining in a mathematical function the value of the age of the subject, the concentration in said blood sample of cytokeratin 18, the fragment M65 or the fragment M30 thereof, and at least two of: the concentration of α-2-macroglobulin in said blood sample, the data representative of liver stiffness and the value of the body mass index of the subject, in order to obtain a score,
c) comparing said score to a predetermined first reference value, and
d) treating the subject having a score that is higher than or equal to said first reference value with an appropriate treatment.

13. The method of claim 1, wherein the treatment that is suitable for alcoholic steatohepatitis includes one or more of cessation of alcohol consumption, a healthy dietary treatment, bariatric surgery and administration a drug.

14. The method of claim 13, wherein the drug is one or more of vitamin E, pioglitazone, liraglutide, semaglutide, obeticholic acid, elafibranor, cenicriviroc, armachol or resmetirom.

15. The method of claim 12, wherein the appropriate treatment includes one or more of cessation of alcohol consumption, a healthy dietary treatment and administration a drug.

16. The method of claim 15, wherein the drug is one or more of vitamin E, pioglitazone, liraglutide, semaglutide, obeticholic acid, elafibranor, cenicriviroc, armachol or resmetirom.

17. The method of claim 9, wherein the first specific time of the disease is before a treatment has started, the second specific time of the disease is during or after treatment.
